(19) United States
(12) Patent Application Publication
(10) Pub. No.: US 2019/0117047 A1
(43) Pub. Date: Apr. 25, 2019

(54) ASSISTANT CLAMPER HAVING LENS

(71) Applicant: EPED Inc., Kaohsiung City (TW)

(72) Inventors: Ta-Ko Huang, Kaohsiung City (TW); Jerry T. Huang, Kaohsiung City (TW)

(21) Appl. No.: 15/910,286

(22) Filed: Mar. 2, 2018

(30) Foreign Application Priority Data
Oct. 19, 2017 (TW) 106135990

(51) Int. Cl. A61B 1/00; A61B 1/06

(52) U.S. Cl. CPC A61B 1/00128; A61B 1/00195; A61B 1/00066; A61B 1/0661; A61B 1/00016; A61B 1/00059

(57) ABSTRACT

An assistant clamper having lens and adapted to clamp at an apparatus is provided, which includes a tube, an optical lens, at least one light emitting element and a working channel. The tube extends along a lengthwise direction. The optical lens is disposed at the tube. The light emitting element is disposed at the tube and located on the same side of the optical lens. The working channel is disposed in the tube and penetrates two opposite sides of the tube along the axial direction of the tube, and the working channel is communicated to an opening portion of a side of the tube, and the apparatus is capable of passing into the working channel through the opening portion.

### FIELD OF THE INVENTION

The present invention relates to an assistant damper and a handle, and more particularly to the assistant clamper having a lens, and the handle combined with the assistant damper.

### BACKGROUND OF THE INVENTION

### 1. Description of the Related Art

As science and technology advance in recent years, many medical apparatuses are equipped with an endoscopy (such as a duct with an optical lens) and provided for entering into a human body to capture images which are used as treatment information. The endoscopy may be used separately or combined with the medical equipment for use. In a medical equipment such as a dental electric burner or suction device for example, since a general dental treatment just needs to observe a patient's mouth through an endoscopy without the need of passing a too-long tube into the patient's body, therefore the tube of the endoscopy is generally provided as a channel for passing the medical equipment and sheathing on the medical equipment. As the medical equipment enters into the patient's mouth, the equipment acts as an auxiliary tool. However, the aforementioned channel generally passes through both front and rear ends of the tube, and the medical equipment needs to pass into the tube from the front end or rear end of the tube, and such operation is relatively inconvenient and unable to fit various types of tubes.

### 2. Summary of the Invention

Therefore, it is a primary objective of the present invention to provide an assistant damper having a lens, and the assistant damper can be clamped onto an apparatus in a simpler and easier manner.

To achieve the aforementioned and other objectives, the present invention provides an assistant clamper having a lens applicable to be clamped to an apparatus, and the assistant damper comprises a tube, an optical lens, at least a light emitting element and a working channel. The tube extends in a lengthwise direction. The optical lens is installed to the tube. The light emitting element is installed to the tube and disposed on the same side of the optical lens. The working channel is disposed in the tube and penetrates both opposite ends of the tube in the axial direction of the tube, wherein the working channel is communicated to an opening portion of a side of the tube, and the apparatus may enter into the working channel from the opening portion.

In an embodiment of the present invention, a part of the tube corresponding to both sides of the opening portion is flexible, and a part of the tube proximate to the surrounding of the optical lens is non-flexible.

In an embodiment of the present invention, the opening portion is parallel to the axial direction of the tube and extended from an end of the tube to the other end of the tube, and the opening portion has a width smaller than the maximum width of the working channel.

In an embodiment of the present invention, the assistant clamper having a lens further comprises a positioning element installed onto the tube and applicable to work with a positioning device to perform positioning.

In an embodiment of the present invention, the positioning element is a positioning probe movably sheathed on the tube and applicable to work with the positioning device to perform a contact positioning.

In an embodiment of the present invention, the positioning element is an infrared element or a light emitting element sheathed on the tube and applicable to work with the positioning device to perform a non-contact positioning.

In an embodiment of the present invention, the positioning element is detachable and may be sheathed on the front of the tube.

In an embodiment of the present invention, the positioning element is detachable and may be sheathed on a side of the tube.

In an embodiment of the present invention, the positioning device is connected online with a registration system for registering positioning information after the positioning device is used.

In an embodiment of the present invention, the assistant damper having a lens further comprises a reflective element installed at the other end opposite to the optical lens of the tube and provided for identifying a relative position of the tube in a positioning process.

In summation, the assistant damper having a lens of the present invention provides the working channel in the tube, and the working channel is communicated to the opening portion on a side of the tube, so that an apparatus can enter into the working channel from the opening portion without the need of passing from an end of the working channel to the other end of the working channel through the tube. In other words, the tube can be sheathed on the apparatus by using the opening portion, so that the apparatus can enter into the working channel, and the assistant damper can be clamped onto the apparatus. Obviously, the assistant damper having a lens of the present invention is applicable to be clamped onto the apparatus with a simple and easy manner.

### BRIEF DESCRIPTION OF THE DRAWINGS

FIG. 1 is a perspective view of an assistant clamper having a lens in accordance with a first embodiment of the present invention;

FIG. 2 is a front view of an assistant damper having a lens as depicted in FIG. 1;

FIG. 3 is a schematic view of an assistant damper having a lens as depicted in FIG. 1 applied to an apparatus;

FIGS. 4 to 7 show a flow chart of positioning an assistant damper having a lens as depicted in FIG. 1;

FIG. 8 is a schematic view of the assistant damper having a lens combined with a positioning component;

FIG. 9 is a schematic view of drawing a positioning figure for the assistant damper having a lens;

FIG. 10 is a schematic view of a positioning figure drawn at the positioning component;

FIG. 11 is a schematic view of the assistant damper combined with a handle;

FIG. 12 is a schematic view of the handle; and

FIG. 13 is a schematic view of a probe combined with the handle.

### DESCRIPTION OF THE PREFERRED EMBODIMENTS

The above and other objects, features and advantages of this disclosure will become apparent from the following detailed description taken with the accompanying drawings.

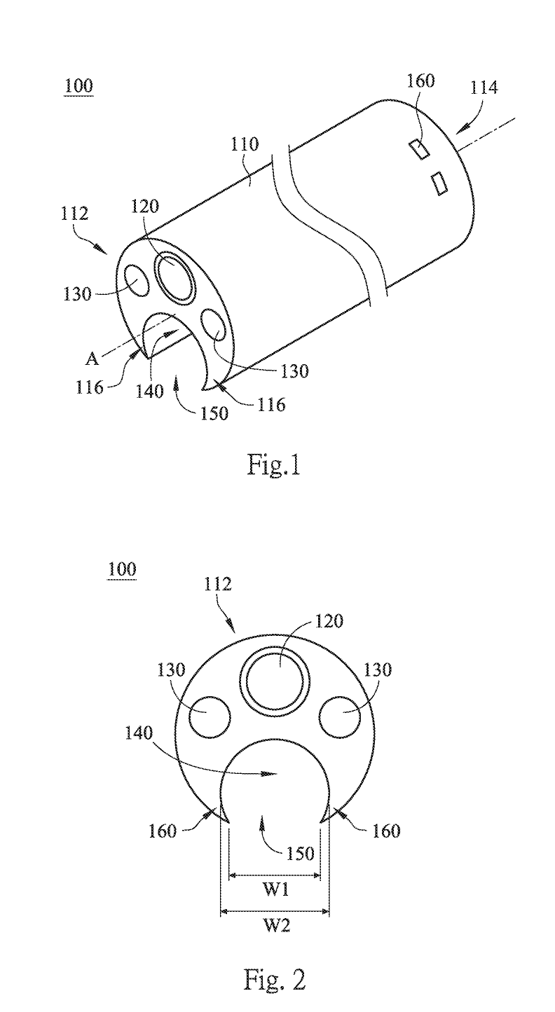

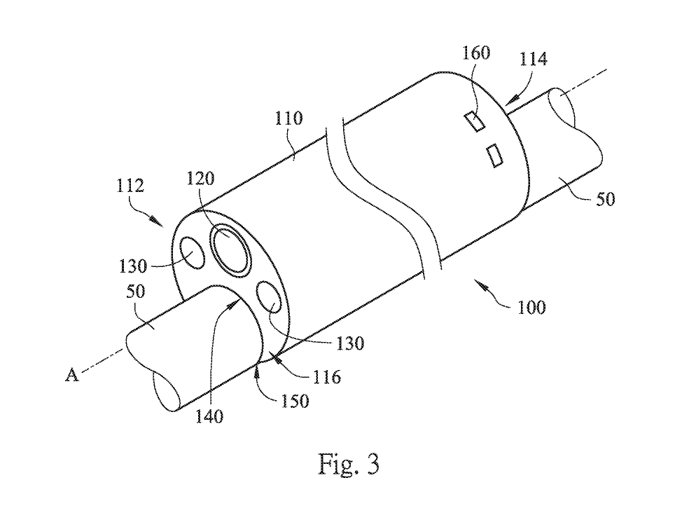

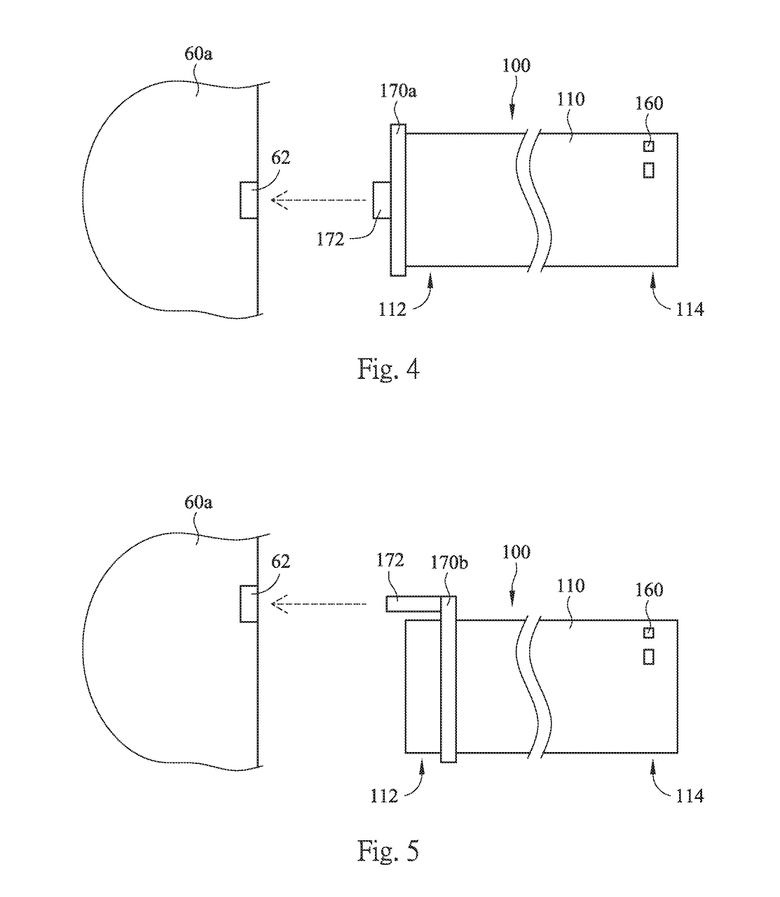

With reference to FIGS. 1 to 3 for a perspective view of an assistant damper having a lens, a front view of the assistant clamper, and a schematic view of the assistant clamper applied to an apparatus in accordance with an embodiment of the present invention respectively, the assistant clamper having a lens 100 is applicable to be clamped to the apparatus 50 such as a medical equipment including an electric burner or suction device (as shown in FIG. 3), so that when the apparatus 50 is used for treatment, the assistant clamper having a lens 100 can capture an image which may be used as the information for assisting the treatment, but the scope of applicability of the present invention is not just limited to such application only.

In this embodiment, the assistant clamper having a lens 100 comprises a tube 110, an optical lens 120, two light emitting elements 130, and a working channel 140. The tube 110 extends in a lengthwise direction (which is the axial direction A of the tube 110) and constitutes the main body of the assistant damper having a lens 100, and the tube 110 has a front end 112 and a rear end 114 opposite to each other. The optical lens 120 is installed to the tube 110 such as a front end 112 of the tube 110. During operation, the tube 110 is entered into a human body) to capture image information. The light emitting elements 130 are installed to the tube 110 and disposed on the same side of the optical lens 120. In other words, the light emitting elements 130 are installed at the front end 112 of the tube 110, so as to provide the lighting required for capturing images by the optical lens 120. It is noteworthy that the quantity, type, and position of the light emitting elements 130 of the present invention are not limited and may be changed as needed. For example, infrared, visible lights, invisible lights, or sound waves used by a device to achieve the positioning effect may be used.

In this embodiment, the working channel 140 is installed in the tube 110 and penetrates through both opposite ends (which are the front end 112 and the rear end 114) of the tube 110 along the axial direction A of the tube. Wherein, the working channel 140 is communicated to an opening portion 150 on a side of the tube 110, and the apparatus 50 may enter into the working channel 140 from the opening portion 150, so that the assistant clamper having a lens 100 is clamped to the apparatus 50. In other words, working channel 140 is penetrated into the rear end 114 from the front end 112 of the tube 110 along the axial direction A. Theoretically, the apparatus 50 may be passed into the working channel 140 from the front end 112 or the rear end 114 of the tube 110, so that the assistant clamper having a lens 100 can be sheathed on the apparatus 50. The longer the tube 110, the more inconvenient operation of sheathing the assistant clamper on the apparatus 50. As a result, the assistant clamper cannot fit various types of apparatuses 50 (such as an apparatus having non-equal widths along the tubular body of the apparatus or having a protrusion). With the design of the opening portion 150 communicated to the working channel 140, the apparatus 50 may not be entered from the front end 112 or the rear end 114 of the tube 110, but may be entered into the working channel 140 from the opening portion 150 instead.

Further, the opening portion 150 of this embodiment is parallel to the axial direction A of the tube 110 and extends from an end of the tube 110 to the other end of the tube 110 (or extends from the front end 112 to the rear end 114) to form a substantially long-strip shape. In addition, the opening portion 150 has a width W1 smaller than the maximum width W2 of the working channel 140 (when the cross-section of the working channel 140 is in a circular shape, the maximum width W2 is its diameter), and a part of the tube 110 corresponding to both sides of the opening portion 150 (which is the clamping portion 116 as shown in FIGS. 1 to 3) is flexible. Preferably, the apparatus 50 has a width smaller than the maximum width W2 of the working channel 140 and greater than the width W1 of the opening portion 150, but the present invention is not just limited to such arrangement only.

Since a part of the tube 110 proximate to the opening portion 150 (which is the clamping portion 116) is flexible, and the opening portion 150 extends along the axial direction A from the front end 112 of the tube 110 to the rear end 114 of the tube 110, so that the apparatus 50 can be entered from the opening portion 150 into the working channel 140 by squeezing the clamping portion 116 to a smaller width W2 while passing from the front end 112 of the tube 110 and existing from the rear end 114 of the tube 110, and then the clamping portion 116 resumes the original position to cover the apparatus 50 by its flexibility, so that the assistant clamper having a lens 100 is clamped to the apparatus 50 as shown in FIG. 3. On the other hand, the part of the tube 110 proximate to the surrounding of the optical lens 120 is non-flexible (or rigid) to prevent the optical lens 120 from being shifted to an unexpected position due to the deformation caused by the flexibility when the tube 110 touches the human body. However, the present invention is not just limited to such arrangement only.

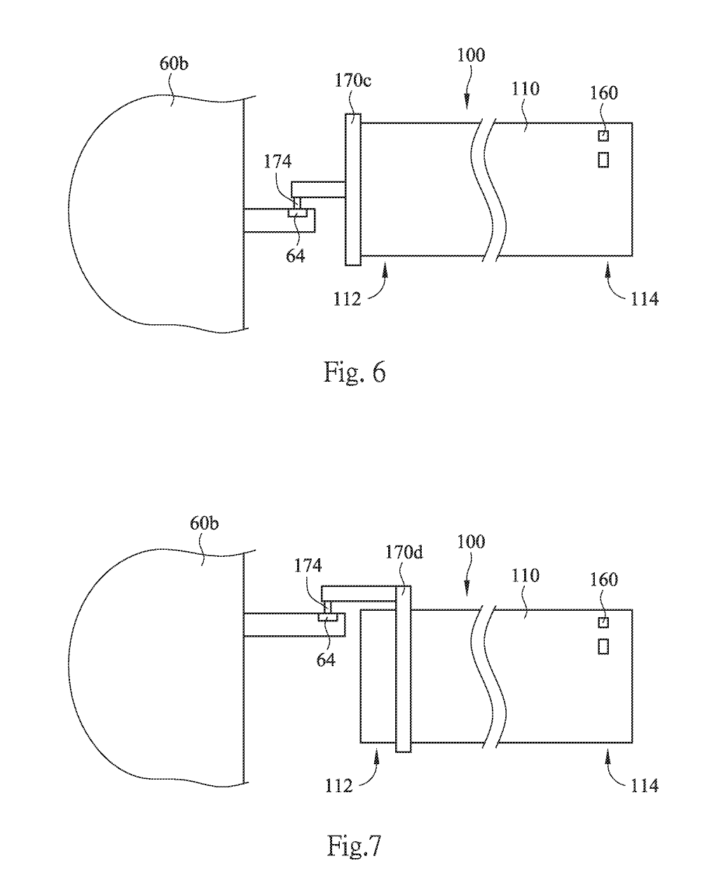

With reference to FIGS. 4 to 7 for the flow chart of positioning an assistant clamper having a lens as depicted in FIG. 1, FIGS. 4 and 7 show that the assistant clamper having a lens 100 of this embodiment is clamped to the apparatus 50 for a related application (such as being clamped to a medical equipment for assisting a treatment), wherein the assistant clamper having a lens 100 is preferably positioned. In other words, before the assistant clamper having a lens 100 is used, the assistant clamper is preferably positioned by a positioning system first and such positioning is used as a reference potion of an image captured by the optical lens 120, and subsequent applications may use to the difference between the image captured by the optical lens and the reference point to find the relative position of the assistant clamper having a lens 100 in a human body.

Specifically, the assistant clamper having a lens 100 of this embodiment further comprises a reflective element 160 installed at the other end of the tube 110 opposite to the optical lens 120 (which is the rear end 114 of the tube 110) and provided for identifying the relative position of the tube 110 in a positioning process. The present invention is not just limited to such arrangement only, but it can be adjusted as needed. In addition, the assistant clamper having a lens 100 further comprises a positioning element installed to the tube 110 and applicable to work with a positioning device for positioning. In other words, the assistant clamper having a lens 100 may work together with the positioning element and the positioning device for positioning and use the reflective element 160 to identify the relative position of the tube 110 in a positioning process (which determines where is the front end 112 or the rear end 114), and also position the relative position of the image captured by the optical lens at such reference point. Wherein, the reflective element 160 may be changed to an active light emitting element. The implementation of the positioning element and the positioning device is described below.

In an embodiment of the assistant clamper having a lens 100 as shown in FIG. 4, the assistant clamper having a lens 100 further comprises a positioning element 170a, such as an infrared element 172 (or a light emitting element) movably sheathed on the tube 110 and applicable to work with a positioning device 60a for a non-contact positioning. For example, a receiving element 62 of the positioning device 60a receives an infrared light and detects its relative position (during the process, the reflective element 160 is used to identify the front end 112 and the rear end 114 of the tube 110). Wherein, the positioning element 170a is detachable (such as an infrared element 172 detachably installed onto a frame) and may be sheathed on the front of the tube 110. In an embodiment as shown in FIG. 5, the positioning element 170b may be an infrared element 172 (or a light emitting element) applicable to work with a positioning device 60a and use its receiving element 62 for a non-contact positioning, wherein the positioning element 170b is detachable (such as an infrared element 172 detachably installed onto the frame) and may be sheathed on a side of the tube 110. Therefore, the way of sheathing the positioning elements 170a and 170b of the present invention on the tube 110 may be adjusted as needed. Preferably, the positioning elements 170a and 170b are detachable, and they can be removed after positioning to minimize the unnecessary contact of components with the human body or the interference of capturing the image by the optical lens 120 after the assistant damper having a lens 100 is clamped to the apparatus 50. However, the present invention is not just limited to such arrangement only.

In this embodiment, the positioning device 60a is connected online with a registration system. After the positioning device 60a is used, the positioning information of the positioning device 60a is registered. In other words, after the positioning element 170a or 170b of an infrared element 172 of the assistant clamper having a lens 100 is working with the receiving element 62 of the positioning device 60a for a non-contact positioning, the positioning information (which is the relative position of the tube 110) will be sent to the registration system for registration to facilitate the next step of the application of the positioning information. For example, when the apparatus 50 is used for treatment, the assistant damper having a lens 100 is positioned by the positioning device 60a in advance, and the positioning result used as a reference point, and the image captured of the optical lens 120 is used together to determine the relative position of assistant damper having a lens 100 in the human body. However, the present invention is not just limited to the aforementioned procedure only.

In an embodiment as shown in FIG. 6, the assistant clamper having a lens 100 further comprises a positioning element 170c such as a positioning probe 174 movably sheathed on the tube 110 and applicable to work with a positioning device 60b for a contact positioning. For example, the positioning probe 174 is in contact with a positioning slot 64 of the positioning device 60b to detect its relative position. Wherein, the positioning element 170c is detachable (such as a positioning probe 174 detachably installed to a frame) and may be sheathed on the front of the tube 110.

In an embodiment as shown in FIG. 7, the positioning element 170d is a positioning probe 174 and applicable to work with a positioning device 60b and uses the positioning slot 64 for a contact positioning, wherein the positioning element 170d is detachable (such as a positioning probe 174 detachably installed to the frame), and may be sheathed on a side of the tube 110. The way of sheathing the positioning elements 170c and 170d of the present invention on the tube 110 may be adjusted as needed. Preferably, the positioning elements 170c and 170db are detachable and may be removed after positioning. However, the present invention is not just limited to such arrangement only.

In addition, the positioning device 60b may be connected online with a registration system. After the positioning device 60b is used, the positioning information may be registered. Such arrangement has been described above and will not be repeated.

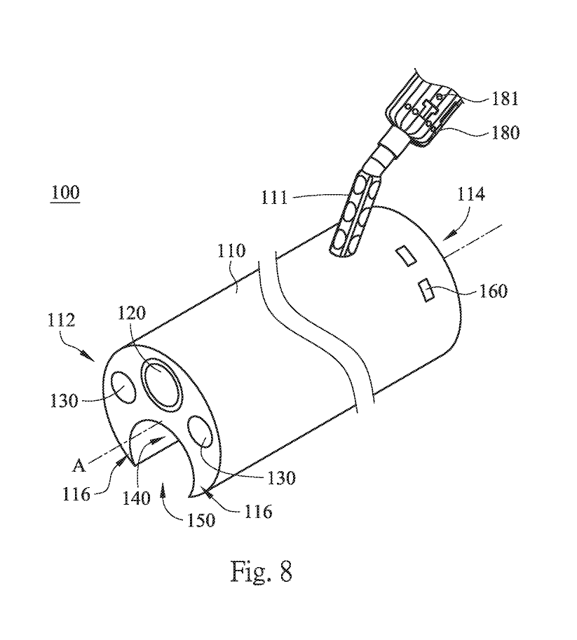

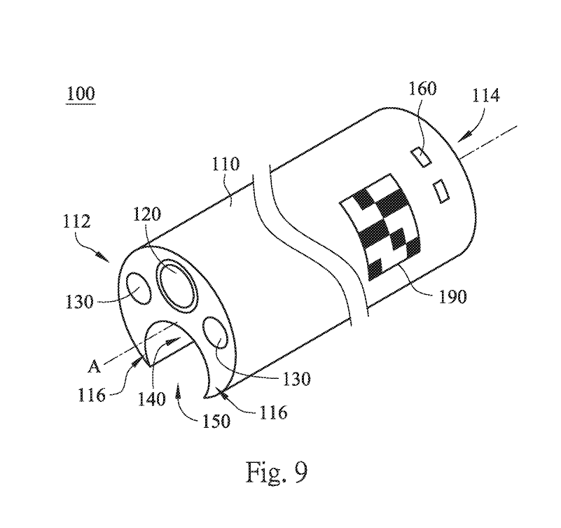

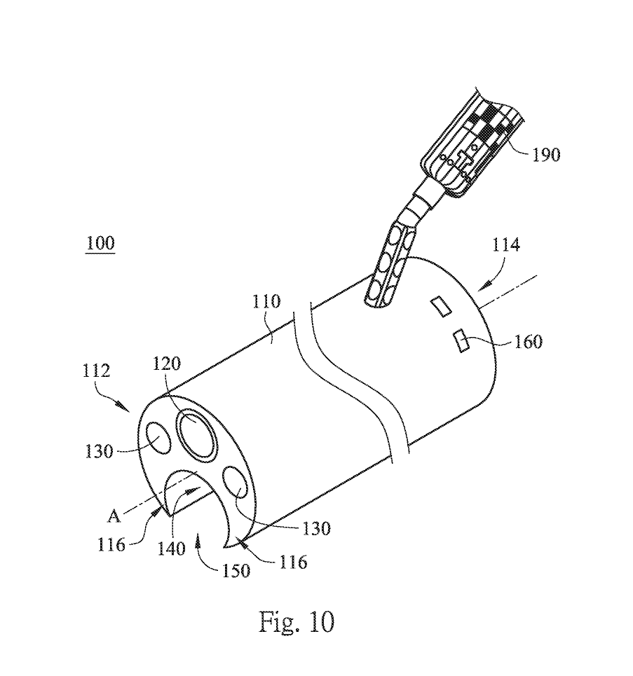

In FIG. 8, a plug rod 111 is installed to the tube 110 and movably coupled to a positioning component 180, and the positioning component 180 has a plurality of reflective elements 181 (or a plurality of active light emitting elements) installed thereto and provided for performing a related position detection. In FIG. 9, the tube 110 has a pattern 190 which is formed by a pattern sticker or a barcode or a plurality of geometrical figures to provide a direction indicating function, and the pattern 190 may be adhered, engraved, or printed onto the tube 110 and provided for the system to have the position detection effect. In addition, the positioning component 180 may have a pattern 190 as shown in FIG. 10 and provided for a system to have the position detection function.

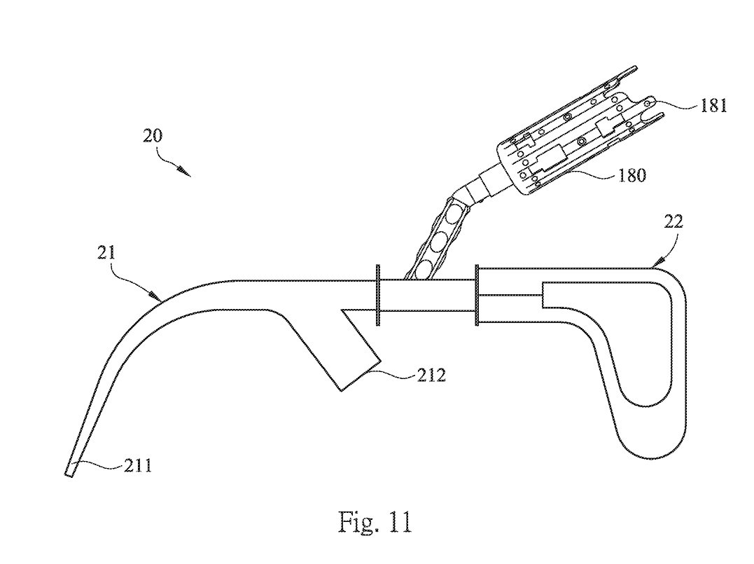

In summation of the description above, the assistant clamper having a lens of the present invention has the working channel in the tube and the working channel is communicated to the opening portion on a side of the tube, so that the apparatus can enter into the working channel from the opening portion without the need of passing from an end of the tube to the other end of the tube into the working channel. Further, the opening portion extends from an end of the tube to the other end of the tube along the axial direction of the tube, and a part of the tube corresponding to both sides of the opening portion is flexible, so that the apparatus can be entered into the working channel from the opening portion by squeezing the flexible opening portion while passing through and extending out from the tube, so that the assistant damper having a lens is clamped to the apparatus. In other words, the tube is sheathed on the apparatus by the opening portion, so that the apparatus can enter into the working channel, and the assistant clamper is clamped to the apparatus. Therefore, the assistant clamper having a lens of the present invention is applicable to be clamped to the apparatus in a simple and easy manner In addition, the tube may be in the shape as shown in FIG. 11, and a curve or a straight line may be formed at the front end to fit various types of apparatuses or meet the requirements for different surgical positions, and the tube may be designed as a hard tube.

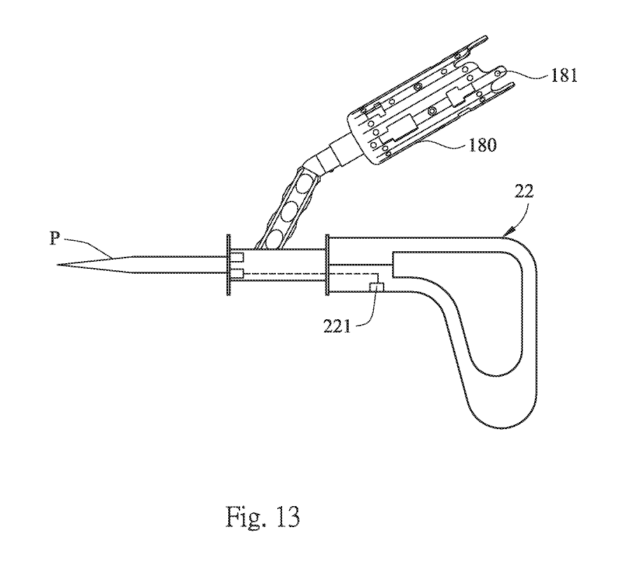

In FIG. 11, a handle 22 has a positioning component 180. The positioning component 180 has a plurality of reflective elements 181 set by a positioning system and provided for the positioning system to capture the position of the reflective elements 181, so as to achieve the effect of the positioning equipment. In addition, the handle 22 may be connected to another apparatus such as a probe as shown in FIG. 13. The handle 22 is provided to achieve the positioning and operation effect and improve the scope of applicability of the handle 22. The positioning component 180 may be movably plugged into the handle 22.

The connection of the handle 22 and the tube 21 includes plugging, latching, screwing, or quick release connection. Since these connections are prior art, they will not be described in details.

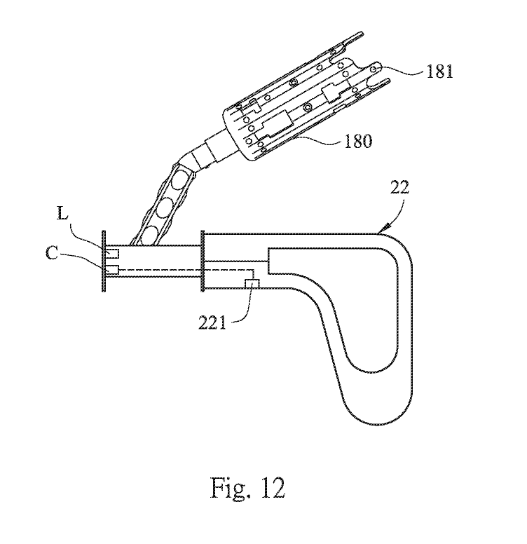

In FIG. 12, the handle 22 further comprises a light emitting source L and a signal port C, and the light emitting source L is configured to be corresponsive to an end of the tube 21 for sending the produced light to the front end through the tube 21, wherein the way of sending the light produced by the light emitting source L to the front end of the tube 21 includes the way through an optical fiber or through a refraction path or a straight path in the tube 21. Since these ways are prior art, they will not be described in details. The tube 21 further has a signal connector (not shown in the figure) configured to be corresponsive to a signal connector of the signal port C and provided for transmitting signals through a contact method. Similarly, this is a prior art, and thus will not be described in details.

Further, the handle 22 has a signal transmission unit 221 coupled to the signal transmission port C via a signal connection, and the signal transmission port C is provided for transmitting a signal to a specified device (such as transmitting a screen of the endoscopy) for storage or a subsequent operation. Wherein, the signal transmission unit 221 transmits information through a wireless transmission means or a cable transmission means.

The assistant clamper having a lens 100 of the present invention may be disinfected directly for a reuse or made of a disposable material. The invention has a wide scope of applicability. In addition, the handle of the present invention has an effect that cannot be achieved by the conventional apparatus. By assembling the handle with various apparatuses for positioning, the invention can be positioned by a positioning system and the position and situation inside the human body can be simulated to improve the assisting effect and the precision of the surgical cutting or other operations. The present invention also overcomes the drawback of the conventional apparatuses just capable of performing an instant position monitoring for specific apparatuses only.

What is claimed is:

1. An assistant damper having a lens, applicable to be clamped to an apparatus, comprising:
a tube, extending in a lengthwise direction;
an optical lens, installed to the tube; and
a working channel, disposed in the tube, and penetrating both opposite ends of the tube in the axial direction of the tube, wherein the working channel is communicated to an opening portion on a side of the tube, so that the apparatus may enter into the working channel from the opening portion.

2. The assistant clamper having a lens according to claim 1, further comprising at least a light emitting element installed to the tube and disposed on the same side of the optical lens, and a part of the tube corresponding to both sides of the opening portion is flexible, and a part of the tube proximate to the surrounding of the optical lens is non-flexible.

3. The assistant damper having a lens according to claim 1, wherein the opening portion is parallel to the axial direction of the tube and extended from an end of the tube to the other end of the tube, and the opening portion has a width smaller than the maximum width of the working channel.

4. The assistant clamper having a lens according to claim 1, further comprising a positioning element installed onto the tube and applicable to work with a positioning device to perform positioning.

5. The assistant clamper having a lens according to claim 4, wherein the positioning element is a positioning probe movably sheathed on the tube and applicable to work with the positioning device to perform a contact positioning.

6. The assistant damper having a lens according to claim 4, wherein the positioning element is an infrared element or a light emitting element sheathed on the tube and applicable to work with the positioning device to perform a non-contact positioning.

7. The assistant clamper having a lens according to claim 4, wherein the positioning element is detachable and may be sheathed on the front of the tube or a side of the tube.

8. The assistant clamper having a lens according to claim 4, wherein the tube has a plug rod provided for movably connecting a positioning component, and the positioning component has a plurality of reflective elements or a plurality of active light emitting elements installed thereto.

9. The assistant damper having a lens according to claim 4, further comprising a pattern disposed on the tube, and the pattern being adhered, engraved, or printed on the tube, and the content of the pattern including a pattern or a barcode or a plurality of geometrical figures for providing a direction indicating function.

10. The assistant damper having a lens according to claim 4, further comprising a reflective element installed at the other end opposite to the optical lens of the tube and provided for identifying a relative position of the tube in a positioning process.

11. The assistant damper having a lens according to claim 1, further comprising a handle movably coupled to the assistant damper, and the handle having a positioning component installed thereto.

12. The assistant clamper having a lens according to claim 11, wherein the handle further has a light emitting source configured to be corresponsive to an end of the tube.

13. The assistant damper having a lens according to claim 11, wherein the handle further has a signal port configured to be corresponsive to the tube and contacted with a signal transmission portion of the tube for a signal connection.

14. The assistant damper having a lens according to claim 13, wherein the handle further has a signal transmission unit installed thereto and coupled to the signal transmission port signal, and a received signal is transmitted to a specific device through the signal transmission port.

15. The assistant damper having a lens according to claim 14, wherein the signal transmission unit transmits information through a wireless transmission means or a cable transmission means.

16. A handle, coupled to the assistant damper having a lens as claimed in claim 1, wherein the handle has a positioning component installed thereto.
